(19) United States
(12) Patent Application Publication
(10) Pub. No.: US 2021/0012264 A1
(43) Pub. Date: Jan. 14, 2021

(54) METHOD AND SYSTEM FOR ESTIMATING HEALTHCARE SERVICE QUALITY

(71) Applicant: Watershed Health LLC, Daphne, AL (US)

(72) Inventor: Arthur G. Grant, III, New Orleans, LA (US)

(21) Appl. No.: 16/508,184

(22) Filed: Jul. 10, 2019

(51) Int. Cl. G06Q 10/06; G16H 10/20; G16H 10/60

(52) U.S. Cl. CPC G06Q 10/06395; G16H 10/60; G16H 10/20

(57) ABSTRACT

The present invention provides a computerized method and system for estimating healthcare service quality for a patient, including generating a communication request for a service provider via a communication platform and requesting a response from the service provider to the communication request. The method and system determines behavior metrics associated with the service provider including a response time value based on a timeliness and a response quality value based on a completeness of the response. The method and system receives a medical care inquiry regarding the patient and therein generates a quality assurance value for the service provider, relative to the medical care inquiry, based on the behavior metrics. The method and system provides, via a user interface, the quality assurance value associated with the service provider.

### COPYRIGHT NOTICE

A portion of the disclosure of this patent document contains material that is subject to copyright protection. The copyright owner has no objection to the facsimile reproduction by anyone of the patent document or the patent disclosure as it appears in the Patent and Trademark Office patent files or records, but otherwise reserves all copyright rights whatsoever.

### FIELD OF INVENTION

The present invention relates generally to healthcare processing networks and more specifically to determining service quality for patient-centered healthcare networks.

### BACKGROUND

There is significant growth in the amount of healthcare related electronic data, such as generated through electronic medical records, electronic devices, and other sources. While the volume of data continues to grow, problems remain in translating this data into actionable results for providing optimal patient care and motivating healthcare providers to provide the highest quality of patient care.

Many of the existing computer processing techniques for improving patient care revolve around analyzing data, but fail to generate actionable results accounting for quality assurance. For example, current techniques seek to create electronic exchanges between various providers within the healthcare continuum, e.g. hospital and physician practices. But these current techniques omit a large portion of care providers, such as skilled nursing facilities, physical therapists, occupational therapists, pharmacies, home healthcare agencies, hospice, emergency medical services, etc.

In recent years, quality data has become available from the Centers for Medicare and Medicaid (“CMS Data”). Studies have determined that CMS Data operates in a purely backwards looking manner and there is poor accuracy for determining future patient quality care based solely on the CMS Data.

One current proposed solution for improving healthcare is described in U.S. Publication No. 2018/0182475 (“475 Publication”), including monitoring various data feeds and using artificial intelligence (“AI”) to detect a significant event or condition. Upon detection, the '475 Publication can then estimate likely next steps and/or generate a course of treatment. This solution is static based on pre-existing medical guidelines, but does not account for patient quality of care or generate a form of quality assurance for the patient.

Another solution is described in U.S. Pat. No. 8,069,080 (“'080 patent), generating healthcare provider quality and cost ratings. Where the '080 patent generates provider quality data, this provider quality is based on the post-care analysis of treatment data. The '080 patent does not account for any real-time or just-in-time post-care or incident inquiries from healthcare providers. Moreover, the '080 patent analysis is independent of any particular patient.

There are techniques for using a wide variety of data sources to generate better or improved data analysis, such as U.S. Publication No. 2018/0121857 (“'857 Publication”). The '857 Publication describes managing healthcare data in a healthcare operating system, the system having multiple individual systems or processing modules. The '857 Publication provides for managing these large networks of data, but like other solutions, does not and cannot provide quality assurance or engender quality of care based decision making, lacking real time or just-in-time data analytics.

Thus, while techniques exist for managing healthcare data from multiple sources, none of these systems can account for or provide quality assurance measures, including motivating healthcare providers to improve quality of care. There exists a need for a method and system that manages healthcare data, estimates quality assurance, and promotes high-quality patient care as part of the patient-centered healthcare computing network.

### BRIEF DESCRIPTION

The present invention provides a computerized method and system for motivating medical care service providers to provide the highest quality of patient care. The computerized method and system operates using a communication platform providing a patient-centered healthcare network. The communication platform collects data from healthcare service providers and patients, facilitates communications across the platform, and allows for referrals between different providers.

The method and system monitors service provider interactions and generates quality assurance values for these service providers. The method and system outputs the estimated quality assurance values within the patient-centered healthcare network. Therefore, estimating and disseminating quality information motivates service providers to provide the highest quality of patient care.

The computerized method and system includes collecting patient and service provider operational data and storing the data within a healthcare database.

The method and system generates communication requests for service providers via the communication platform. The request can be generated by the system itself or a service provider uses the platform to communicate with another service provider. The method and system requests a response from the service provider. In collecting operational and interaction data, the method and system, determines behavior metrics associated with the service provider.

Behavior metrics include a response time value based on a timeliness of a response by the service provider to the communication request and a response quality value based on a completeness of the response by the service provider to the communication request. By way of example, if the service provider submits a full and complete response with a quick response time, this value is different from a provider that fails to answer or delays a response and provides an incomplete response.

The communication request can be one or more questions. The communication request can be a data request, for example seeking medical records or other patient data.

The communication request can be a simple question or set of questions relating to a particular medical care incident. By way of example, an incident can include a routine medical event such as a patient operation or medical discharge, or an event can be anomalous event, for example a hospital re-admission or emergency room visit.

The communication request can also include questions relating to the medical care incident, questions allowing for root-cause analysis of the incident.

As part of the patient-centered healthcare network, the method and system includes receiving a medical care inquiry regarding the patient. For example, an inquiry can be seeking placement within a residential care facility, requesting information on potential surgeons for a specific surgery, requesting information on potential physical therapists, etc.

The method and system therein generates a quality assurance value for the service provider, relative to the medical care inquiry, based on the behavior metrics. Via a user interface, the method and system therein provides to the patient the quality assurance value associated with the service provider. The presentation of this quality assurance value can be used as part of a service provider networking system for recommending or referring additional services providers to the patient.

The method and system therein motivates services providers to provide the highest quality of patient care including timely attending to medical care inquiries within the central communication platform.

The communication platform provides just-in-time communication between service providers. This communication allows for coordination of care. This communication also allows for root-cause analysis of events. As service providers improve responsiveness within the system, the assurance of quality care increases.

The method and system provides a patient-centered healthcare network. Service providers engage the network for regular interactions, including entering operational data relating to day to day services. The network stores that data in the central database. The method and system can then additionally generate the medical care quality value also based on this operational data. The method and system may also collect and use patient data as part of the quality assurance value determinations.

Thereby, the method and system estimates healthcare service quality via information collected and exchanged within the patient-centered healthcare network.

### BRIEF DESCRIPTION OF THE DRAWINGS

FIG. 1 illustrates a block diagram of a processing system for generating healthcare recommendations;

FIG. 2 illustrates a block diagram of the patient-centered healthcare network including data collection and exchange operations;

FIG. 3 illustrates one embodiment of the processing system of FIG. 1;

FIG. 4a illustrates a flowchart of steps of a method for estimating healthcare service quality;

FIG. 4b illustrates a flowchart of further steps of the method for estimating healthcare service quality;

FIG. 5 illustrates a flowchart of the steps a method for estimating healthcare service quality in response to a medical incident;

FIG. 6 illustrates a flowchart of the steps of a method for estimating healthcare service quality based on a communications between service providers;

FIG. 7 illustrates a screenshot of engagements with a service provider;

FIG. 8 illustrates a screenshot of generating a medical inquiry; and

FIG. 9 illustrates a sample screenshot of a provider recommendation.

A better understanding of the disclosed technology will be obtained from the following detailed description of the preferred embodiments taken in conjunction with the drawings and the attached claims.

### DETAILED DESCRIPTION

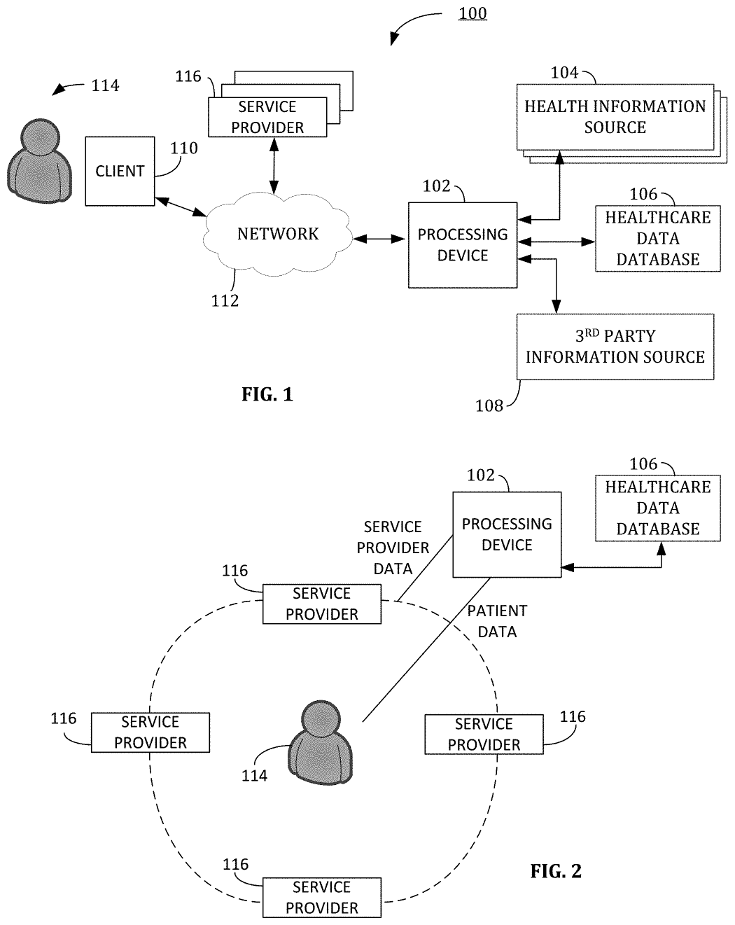

FIG. 1 illustrates a block diagram of a system 100 for facilitating data exchange, enabling multi-party communication and estimating healthcare service quality for patient. A patient may be any person or persons requiring or seeking medical care information. The term patient may specifically refer to the person needing the medical assistance, but can also refer generally to a person and family, care providers, or any other assistant or support structure relating to medical care.

The processing system 100 of FIG. 1 illustrates a processing device 102 accessing external data sources. In the embodiment of FIG. 1, the sources include health information sources 104, healthcare network database 106, and third party information sources 108. These sources may be locally stored and connected, but also may be network connected, such as located in a cloud-based computing or networking system. Additionally, these sources can be public or proprietary data sources.

The processing system 100 operates a software platform enabling service providers to easily practice three important behaviors in clinical care with other members on the network: communication; data exchange; and quality assurance. The system 100 measures the behaviors of the healthcare providers and therein estimates quality assurances of service based on behavior metrics. The system 100 allows for patients and providers to electronically build patient-centered healthcare networks using real-time metrics to guide decision making.

In the system 100, the processing device 102 engages a client device 110 via the network 112, providing the interface for a user 114. The processing device further engages service providers 116. Service providers 116 may be any individual or organization providing healthcare or related services, including for example, but not limited to, hospitals, pharmacies, emergency medical services, residential care facilities, individual doctors/nurses.

While not illustrated, the service providers 116 further include user interface functionality for communicating with the processing device 102. Communications with the processing device 102 can include sharing operational data, interacting with patient records, submitting patient information, communicating with other service providers or patients via the platform, by way of example.

The processing device 102 may be any suitable device or devices for performing processing operations as described herein. The processing device 102 may be a unitary processing device or system or can be a distributed processing system, such as one or more processing networks disposed in a networked or cloud based computing platform. As described in further detail herein, the operations of the processing device are performed in response to executable instructions from one or more computer readable medium, such as a memory device having the instructions stored therein.

The health information source 104 can be any suitable source for health information or health related data, such as locally or networked sources, including public and private data sources. By way of example, health information source 104 can be electronic medical records. In another example, the source 104 can be a medical billing and/or insurance data. In another example, the health information source can include CMS Data or similarly generated qualitative data.

Another sample of a source 104 can be information about medical doctors or medical facilities, such as assembled by external or monitoring sources. For example, for medical facilities may track patient care data such as average length of stay for specific procedures, re-admission rates, patient demographic data, etc.

The healthcare network database 106 includes stored data generated within the patient-centered healthcare network via the processing device 102. As service providers 116 engage the processing device 102, they submit data and those interactions are then stored within the database 106. For example, service provider 116 can enter operational data relating to day to day operations, including interactions with patients.

Similarly, as users interact with the processing device 102, such as submission of health data or other interactions, that information is additionally stored in the database 106.

Third party information source 108 can be any other source not categorized as a health information source 104. The third party information source 108 can be any other suitable information usable for assisting in or being a computational factor in the processing operations. By way of example, one source 108 may be map data for providing distances, such as an estimated distance between a residential care facility and the patient's home address (e.g. family). In another example, one source 108 can be weather information predicting bad weather (e.g. snowstorm, hurricane, etc.).

The client 110 may be any suitable processing device or devices operative for engaging the user 114. The client 110 may include dedicated or installed executable software for local execution, such as a local downloadable application. The client 110 may utilize any suitable browser application and run the browser as the processing gateway for network-based functionality. The client 110 can be a stationary device, such as a desktop computer, or can be a mobile computing device having interconnectivity to the processing device 102.

The network 112 can be any suitable public or private network, or a combination thereof. In one embodiment, the network 112 is the Internet, but may also include local area networks and/or mobile networks facilitating engagement and communication thereacross.

The user 114 can be one or more users engaging the client device 110 for seeking a healthcare recommendation. For example, the user 114 can be a hospital administrator working with the patient for providing the recommendation or referral. Similarly, the user 114 can be the patient, a guardian, caretaker, etc. The user 114 may also be a service provider seeking a recommendation or referral for another service provider. As used herein, any referral or related output can include necessary statement or precautions ensuring compliance with regulatory and anti-kickback regulations.

Within FIG. 1, the service provider 116 can also be a person, facility, organization, company, producer, insurer, or other entity that provides medical or medical-related services. The service providers 116 interacts with the processing device 102 via the network 112.

As illustrated in FIG. 1, the processing device 102 engages any number of health information sources 104, the database 106, third party information sources 108, as well as communicating with any number of service providers 116.

The system 100 uses the processing device 102 to estimate the healthcare service quality based on service provider 116 behavior metrics.

FIG. 2 illustrates a block diagram of the patient-centered healthcare network. The processing device 102 runs the communication platform engaging with users 104 and service providers 116, storing data in the database 106.

The service providers 116 run and manage operations using the communication platform. By way of example, a service provider can manage a patient visit, such as scheduling the visit, accessing medical records, processing patient intake, processing billing, recording and updating medical records from visit, and patient follow-up post-visit.

As the service provider uses the processing device 102, the exchange of data is stored in the database 106. Data can be relative to patients, but data can also be service provider day-to-day operations. Data stored in the database 106 can include, but not limited to, clinical behavior data, communication behavior, data sharing behavior, and quality assurance behavior.

Similarly, the database 106 can store patient data. Patient data may include, for example, demographic information, hospitalization information (admission, discharge, transfer information), medical and related documents, medication information, vital sign information, and test result data. In some embodiments, the patient may submit this information directly, such as submitting documents and filling out a medical background survey, intake form. In another embodiment, the data may be collected from other sources, such as wirelessly connected devices for example a pedometer measuring exercise, a heart rate monitor, etc.

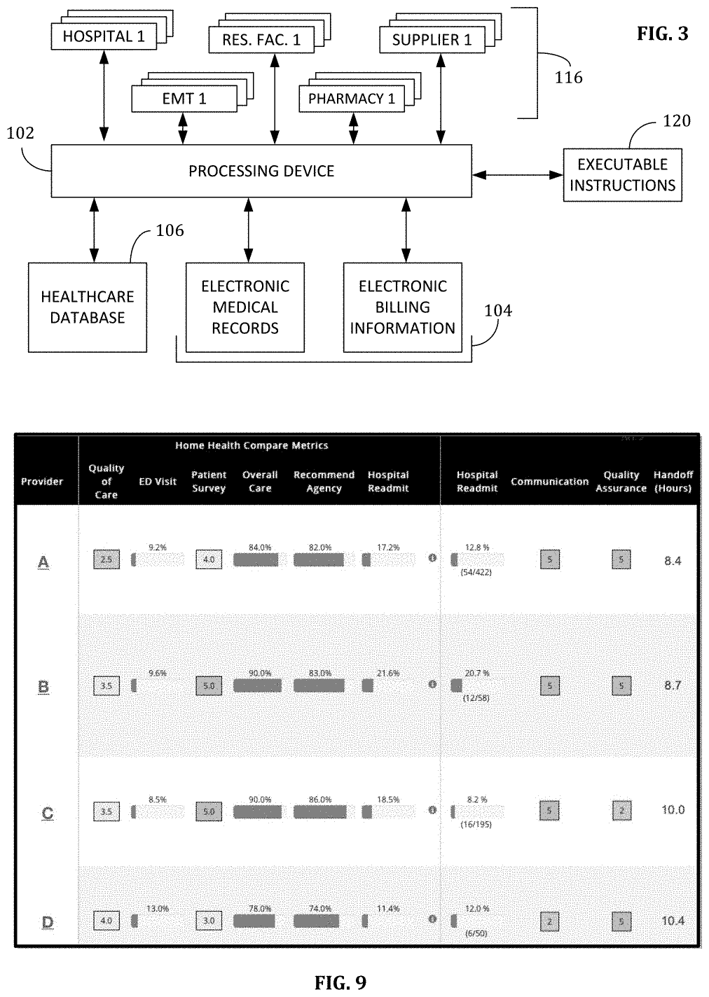

FIG. 3 illustrates a further embodiment of part of the system 100 of FIG. 1. The processing device 102 engages a plurality of service providers 116. The processing device 102 receives executable instructions stored in a computer readable medium 120.

The processing device 102 can include user and service provider registrations, as well as HIPPA compliance, data encryption and secure handling, as well as other recognized system safeguards.

FIG. 3 illustrates numerous exemplary service providers 116 engaging the processing device 102. The listed examples of service providers include various hospitals, emergency medical transport services, residential care facilities, pharmacies, and suppliers. These are exemplary providers and FIG. 3 is not a restrictive or exhaustive list.

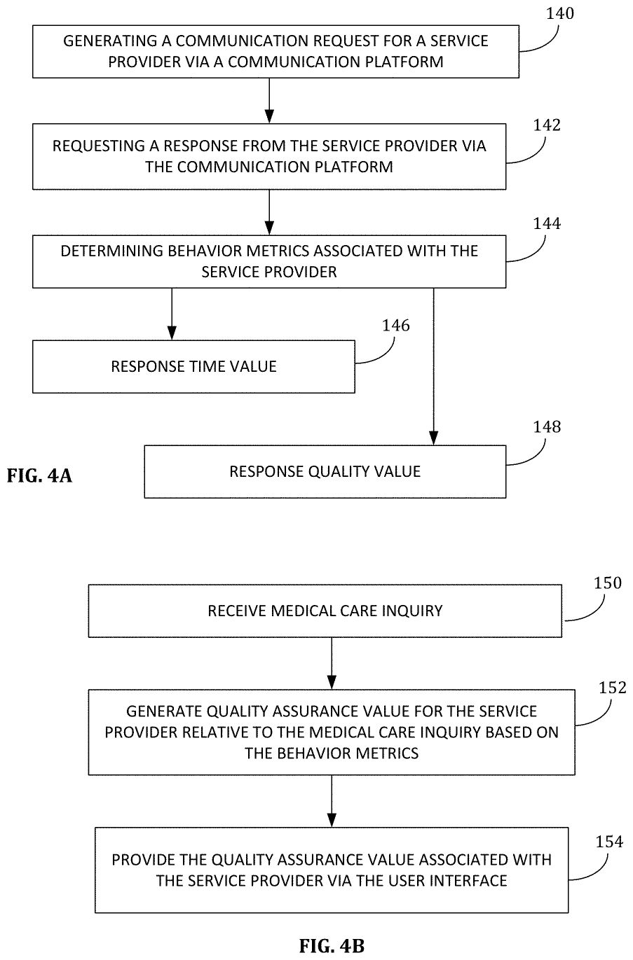

FIG. 4a illustrates a flowchart of a method for estimating healthcare service quality, using the system 100 of FIG. 1. Step 140 is generating a communication request for a service provider via the communication platform. The request can be any suitable type of request, including request for communication, data exchange, and provider behavior.

Step 142 is requesting a response from the service provider to the communication request. In one embodiment, the request of a response may be based on a notification requesting a response. In another embodiment, the request of the response can be based on predetermined operations by the service provider, for example when seeing a patient, the service provider should then enter patient medical information.

Step 144 is determining behavior metrics associated with the service provider. Determining these behavior metrics is based on tracking interactions by the service provider with the communication platform.

The first behavior metric is determining a response time value, step 146. The second behavior metric is determining a response quality value, step 148.

The timeliness relates to how quickly the service provider responds to the inquiry, such as tracking within the context of hours or days. The completeness of the response relates to addressing all the questions and providing complete responses.

Timeliness, as used herein, relates to the amount of time between two measurable events. For communication, timeliness can be measured as the elapsed time between submission of an inquiry to the service provider and response receipt. For data exchange, timeliness can be measured as the elapsed time between when a data request is made to the service provider and when the requested data is provided. For clinical behavior, timeliness can be measured as the elapsed time between one clinical event, such as hospital discharge, and another clinical event, such as a setting a follow-up office visit. For quality assurance, timeliness can be measured as the elapsed time between when a request to perform root-cause analysis of an event is made to the service provider and when the service provider answers questions allowing for the root-cause analysis to be performed.

Timeliness can be a defined scale as predetermined by the system or a system administrator. By way of example, a response within the first 60 minutes can receive a top score, with the score being reduced for future timed interval (e.g. 120 minutes) during normal business hours. Timeliness can also be a relative scale predicated on responsiveness of other service providers. For example, if the average response time is 6 hours, a response sooner than 6 hours can have a higher rating and a response time longer than 6 hours can have a lower rating.

Similarly, completeness can be a relative or preset scale. One technique for completeness can be indicating if an answer is received. For example, the inquiry may include yes/no questions and monitor receipt of the response. In this example, the completeness is not predicated on a type of answer, rather the actual submission of an answer, any answer. In another example, for open-ended questions, completeness may include any other suitable value such as counting the number of words of an answer, where a three word answer can indicate an incomplete response but a twenty-five word answer indicates a completed response.

FIG. 4a relates to collecting these behavior metrics. By way of example, the steps of FIG. 4a can be repeated for each communication request, the behavior metrics being collected over a period of time.

By contrast, FIG. 4b illustrates steps using the collected behavior metrics for estimating healthcare service quality. Step 150 is receiving a medical care inquiry, relating to a patient or a user request. For example as illustrated in FIG. 1, the user 114 may access the processing device 102 via the client device 110, requesting a medical service. The medical care inquiry, being related to a patient, can also be submitted by a service provider, for example a service provider seeking another service provider to provide additional resources or services to the patient.

For example, the medical care inquiry can be to request medical discharge from a hospital and placement in a residential facility. This inquiry can include one or more service providers, e.g. medical transport services, residential facility services, pharmacy services, physical therapy services, etc.

With these services, there are associated attributes. These attributes can be any suitable attributes as recognized by one skilled in the art. The attributes can relate to quality of care and patient safety. In the above example of medical transport services, attributes can include availability of vehicles, driver safety record, etc. In the above example of residential facility services, the attributes can include nurse-to-patient ratios, hospital re-admission rate, distance to emergency room, etc.

Step 152 is generating a quality assurance value for the service provider relative to the medical care inquiry, which can be based on the behavior metrics. The responsiveness of the service provider to response requests from the communication platform indicates higher quality of service provider attention to detail. Therefore, these behavior metrics are used to generate the quality assurance values.

In one example, the medical care quality value can be based on the comparison or scaling of the service providers values relative to similarly-situated service providers. In another example, the medical care quality value can be a cumulative value for the service provider based on the service provider interactions as measured in the patient-centered healthcare network.

It may be presumed that if a service provider has a high rating relative to its responsiveness to the response request, this indicates a high level of overall quality. Therefore, where the user or patient may be seeking quality assurance for a service provider providing service A, e.g. timeliness of medical transport, the provider's high ranking can indicate reliability in service B, e.g. having readily-available medical services during transport.

Step 154 is providing the quality assurance value associated with the service provider, estimating healthcare service quality for the patient. This value is presented via a user interface accessing the communication platform, such as via a user interface running on the client device 110 accessing the processing device 102 via the network 112 of FIG. 1. In another example, the quality assurance value can be provided to another service provider via the user interface.

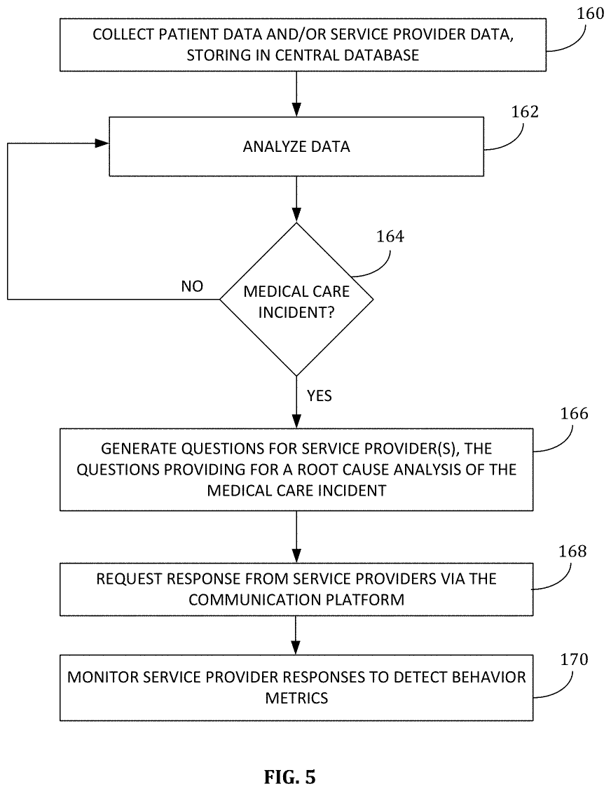

FIG. 5 illustrates a flowchart of steps of one embodiment of estimating healthcare service quality based on communication based on a medical care incident. Step 160 is collecting patient data and service provider data, as well as storing said data in a central database. As noted above, the patient data can be any suitable data relating to the patient and the service provider data can include operational data, as well as any other related data.

Step 162 is to analyze the data. Analysis includes step 164, determining if a medical care incident occurs. A medical care incident can be an anomalous event, for example a negative event. The event can occur with a patient or can be more general. In one example, an event can be a patient being re-admitted to a hospital within thirty days of hospital discharge. Monitoring patient data can detect a hospital discharge and a hospital re-admission occurring within the defined time period. Moreover, an anomalous event does not expressly indicate or imply a negative occurrence, but can include general standard of care inquires.

In another embodiment, a medical care incident may be based on user or service provider input. For example, an administrator may manually enter an event or a doctor may request investigation to assist in further medical care for the patient.

If detected, step 166 is to generate questions for service provider(s) relating to the medical care incident. In one embodiment, the questions may be directed for providing a root cause analysis of the incident

In one embodiment, the method may determine all connected service providers associated with the event. This determination may be performed using the patient data as well as other data. Similarly, the determination may be performed by an administrator or other user manually selecting or designating service providers.

Maintaining the above example of hospital re-admission within a preset time from discharge, the questions may be standard questions relating to the patient's health and monitoring of health prior to discharge. The service provider here may be the hospital facility, nurses, and doctors associated with the patient. Another service provider may be the pharmacy if prescriptions were issued.

In one embodiment, the system may include pre-generated questions relating to an incident. Questions can include standard questions relating to the service providers role in the incident. In another embodiment, an external engine such as an artificial intelligence engine, can generate specific questions for the various service providers.

Step 168 is to request a response from the service provides via the communication platform, similar to step 142 of FIG. 4 above. Step 170 is monitoring the service providers responses to detect behavior metrics, similar to steps 144-148 of FIG. 4 above.

Therein, the method monitors how service providers respond to requests relating to medical care incidents. The timeliness and completeness of the response again translates to assurance of quality, e.g. attention to detail, by the service provider. Thus, this method provides additional metrics for generating quality assurance values for service providers.

Another embodiment is monitoring operational data for detecting behavior metrics associated with the standard data entry. Under normal operations, the service providers should be entering this operational data in a regular and timely manner. This regular and timely (and complete) data entry indicates a higher degree of quality of care by the service provider.

As used herein, operational data can relate to operational features of the service provider engaging in its normal practices. Operational features can include the elapsed time between two events (e.g., hospital discharge and a post-discharge office visit) when that information is either manually entered into the system or electronically fed into the system via some type of electronic interface. It can also include the completeness of doing some measurable task (e.g., administering an insulin injection every day) when that information is either manually entered into the system or electronically fed into the system via some type of electronic interface. The platform, colleting this operational data, therein monitors the timeliness and completeness of these activities by the service providers. This data is already being collected, which now is usable for estimating quality assurance.

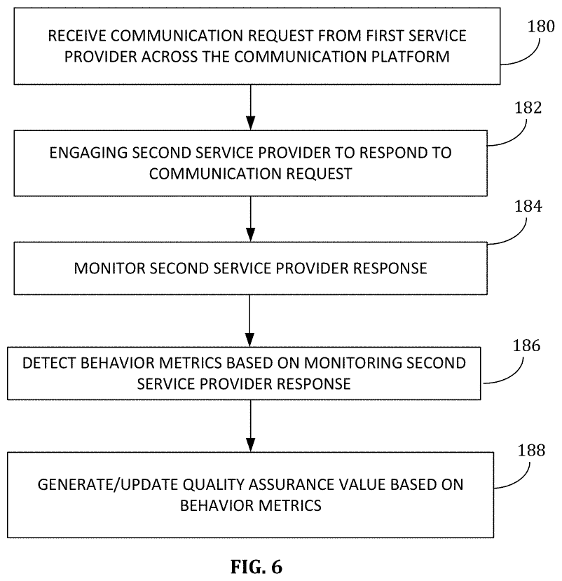

FIG. 6 illustrates another manner of service provider behavior, responding to communication requests across the communication platform. Service providers can communicate across the platform, including requesting information and/or data from other service providers. For example, a first service provider can request patient information or submit questions, for example requesting or asking about a patient's prescribed medicines from a second service provider. In another example, the first service provider might request patient records, x-ray image files, or other type of data. How the second service provider responds to those requests can indicate quality assurance.

Step 180 is to receive a communication request from a first service provider. One example can be a patient having a consultation with the first service provider, and the patient's regular doctor is the second service provider. The first service provider can request information on the current medications, including timing and dosage. This request is processed across the communication platform.

Step 182 is engaging the second service provider to respond to the communication request. Step 184 is then monitoring the second service provider response, including if/when the service provider responds to the inquiry.

Step 186 is detecting the behavior metrics therefrom. For example, if the second service provider quickly and completely answers the inquiry, this can indicate a high quality assurance value. Similarly, if the second service provider quickly responds, but gives incomplete information, e.g. provides prescription dosage information but omits timing information, or fails to timely respond, this can indicate a lower quality assurance value.

Step 188 is to generate and/or update quality assurance values based on the detected behavior metrics. Step 188 can therein provide information usable for presentation to a patient, user, or other system operator seeking quality assurance information.

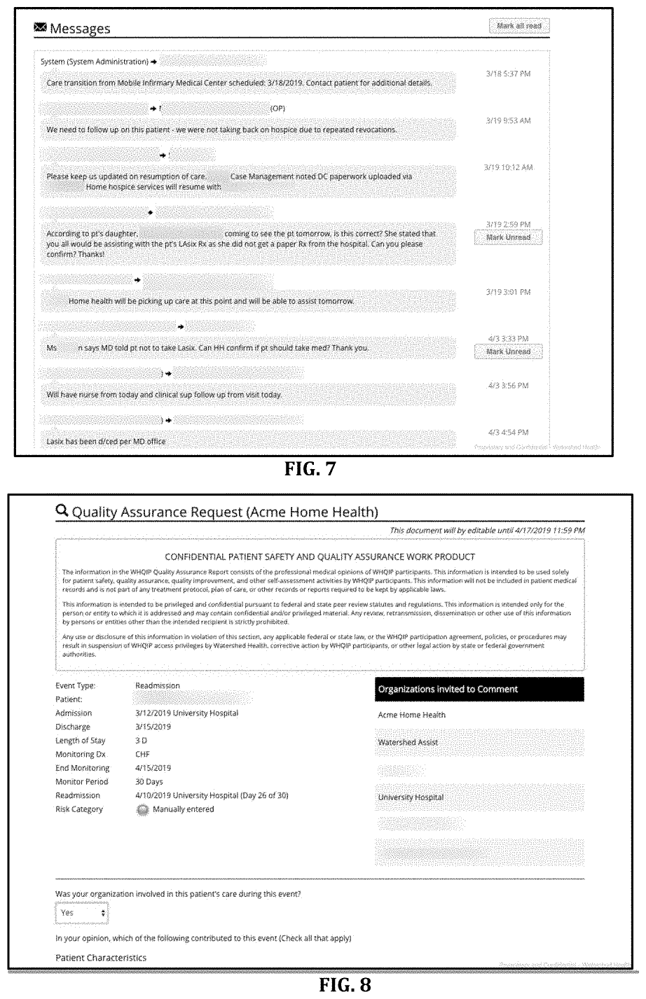

For further examples of the user interface and service provider interactions, FIG. 7 is a sample screenshot of an inquiry to the service provider via the user interface. Similarly, FIG. 8 is a sample screenshot of a user interface screen for generating a medical care inquiry.

FIG. 9 is a sample screenshot of an output display including the ranking of service providers. Each service provider (A-D) has various metrics with associated values. The ranking of the providers generates the recommendation. In this sample screenshot, the system metrics including both a quality assurance metric and a communication metric. Where provider A and provider B has identical quality assurance values (“5”), provider A includes a higher communication factor and is thus recommended higher within the display rankings.

In another embodiment, the method and system may additionally use the third party information sources for assisting in recommendations. As described above, these sources can be any information usable for facilitating an informed decision by the patient or administrator. One example noted above is map data including mileage. The method and system can then update or modify recommendations based on this map data. For example using the FIG. 7 screenshot, if Provider A was located 25 miles from the patient's family and Provider B was located 2.5 miles, the inclusion of mileage may change the listed order of providers. In this case, the ease of family access can directly relate to the ability for the patient to be visited by family, improving overall mood for the patient and can thus increase quality of medical care.

In one embodiment, the healthcare management system described herein can include user interface functionality for the user to include or remove these third party factors as requested for improving search results. Again using the distance example, if the patient does not have family in the area, the distance between the facility and the patient's home can thus be ignored or its value reduced.

In another embodiment, the system may additionally use CMS Data as additional input for estimating healthcare service quality.

The above embodiments relate to a service provider seeking a referral and using the quality assurance value as part of the referral analysis. A further embodiment can include a service provider using the quality assurance value of another service provider in deciding to accept a referral.

For example, service provider A seeks to refer a new patient to a service provider B, but service provider B is unsure about accepting this new patient. Service provider B can review the quality assurance value of service provider A. In this example, if service provider A has a low quality assurance value, service provider B may not want the referral because of concerns about timely receipt of responses to communications about the patient. By contrast, if service provider A has a high quality assurance value, service provider B can accept the referral with greater confidence.

The method and system, operating within the patient-centered healthcare network, motivates service providers to provide the highest quality of services. The system and method generates ranking of service providers recommendations based on the information collected within the system.

The system monitors, records, and tracks daily on-going transactions with patients and service providers. The system records just-in-time activities, logging interactions into the central platform.

When an event occurs, the system can detect the event and inquire with various service providers as to determine potential causation. The method and system can therein provide root cause analysis of why things go wrong. Part of that analysis includes providers providing complete and timely responses to the inquiries.

As providers improve responsiveness to medical care inquiries, the providers quality assurance value increase. The service provider being responsive to the inquiries is a key indicator of service quality and usable for generating the medical care quality value and subsequent rankings.

The method and system estimates healthcare service quality by monitoring the service providers. Therein, the system and method motivates service providers to provide not only the highest quality of care, but be additionally responsive to the inquiries to improve quality rankings within the patient-centered healthcare network.

The method and system provides a software platform that facilitates inter-provider communication (including root-cause analysis) and data exchange. The method and system quality assurance because increased communication and data exchange leads to better service provider quality.

In the method and system, service providers can use these behavior metrics to inform choices about building patient-centric care networks. Making informed decisions based on quality assurance values associated with service providers will be, by definition, make better networks for the user/patient.

The system and method is additionally usable by service providers. They can gain insight about their own strengths and weaknesses, such as determining or viewing their own quality assurance values. The service providers can use these quality assurance values to inform business decisions such as whether to enter a risk-based contract with another provider. Similarly the method and system can be used to inform patient-level decisions such as choosing the best performing provider (e.g., best nursing home) when building a patient-centered care network.

The method and system further enables the referral of patients from one provider to another using the system. The system-generated metrics are seamlessly integrated into this referral process by presenting metric information to system users during the provider selection process. The referral process can be predicated on assurance values, as well as other data, operating in compliance with all required laws, including anti-kickback laws.

The method and system therein improves patient care. Service providers have a natural incentive to obtain better metrics in order to receive more referrals. Within this system, better metrics are obtained by optimizing communication, data exchange as well as certain measured provider behaviors.

FIGS. 1 through 9 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. Moreover, Applicant does not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments so fully reveals the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein.

1. A computerized method for estimating healthcare service quality for a patient, the method comprising:
detecting a medical care incident associated with a service provider;
generating a communication request for the service provider, the communication request including a plurality of questions relating to the medical care incident;
requesting a response from the service provider to the communication request via the service provider addressing the plurality of questions via the communication platform;
electronically determining behavior metrics associated with the service provider including a response time value based on a timeliness of the response by the service provider to the communication request and a response quality value based on a completeness of the response by the service provider to the communication request;
receiving a medical care inquiry regarding the patient, the medical care inquiry requesting medical services for the patient;
accessing a third party data database and retrieving third party information therefrom;
electronically generating a quality assurance value for the service provider, relative to the medical care inquiry, based at least on the behavior metrics;
electronically generating a recommendation relating to the service provider based on the quality assurance value and the third party information; and
transmitting the recommendation including the quality assurance value associated with the service provider, the recommendation visible within a user interface executed on a third party processing device.

2. The method of claim 1, wherein the service provider is a first service provider and the communication request includes an electronic question submitted to the first service provider across the communication platform by a second service provider.

3. (canceled)

4. The method of claim 1, wherein the plurality of questions provide for a root cause analysis of the medical care incident.

5. The method of claim 4, wherein the medical care incident is a negative event relating to medical care.

6. The method of claim 1 further comprising:
receiving operational data from the service provider based on the service provider entering the operational data via the user interface, the operational data relating to operations of the service provider providing healthcare services, wherein the electronically generating the quality assurance value for the service provide is also based on the operational data.

7. The method of claim 1 further comprising:
collecting patient data from a plurality of data sources and storing the patient data within a healthcare database, wherein one of the data sources is an electronic medical record of the patient; and
generating the quality assurance value also based on the patient data.

8. (canceled)

9. A system for generating healthcare recommendations for a patient, the system comprising:
a computer readable medium having executable instructions stored therein; and
a processing device disposed within a central communication platform, the processing device, in response to the executable instructions, operative to:
detect a medical care incident associated with a service provider;
generate a communication request for the service provider, the communication request including a plurality of questions relating to the medical care incident;
request a response from the service provider to the communication request via the service provider addressing the plurality of questions via the communication platform;
determine behavior metrics associated with the service provider including a response time value based on a timeliness of the response by the service provider to the communication request and a response quality value based on a completeness of the response by the service provider to the communication request;
receive a medical care inquiry regarding the patient the medical care inquiry requesting medical services for the patient;
accessing a third party database and retrieving third party information therefrom;
generate a quality assurance value for the service provider, relative to the medical care inquiry, based at least on the behavior metrics;
generate a recommendation relating to the service provider based on the quality assurance value and the third party information; and
transmit the recommendation including the quality assurance value associated with the service provider, the recommendation visible within a user interface executed on a third party processing device.

10. The system of claim 9, wherein the service provider is a first service provider and the communication request includes an electronic question submitted to the service provider across the communication platform by a second service provider.

11. (canceled)

12. The system of claim 9, the processing device further operative to:
receive operational data from the service provider based on the service provider entering the operational data via the user interface, the operational data relating to operations of the service provider providing healthcare services, wherein the electronically generating the quality assurance value for the service provide is also based on the operational data.

13. The system of claim 9, the processing device further operative to:
collect patient data from a plurality of data sources and storing the patient data within a healthcare database, wherein one of the data sources is an electronic medical record of the patient; and
generate the quality assurance value also based on the patient data.

14. A computerized method for estimating healthcare service quality for a patient, the method comprising:
detecting a medical care incident associated with a plurality of service providers;
generating a communication request for the service providers, the communication request including a plurality of questions relating to the medical care incident,
requesting a response from each of the plurality of service providers to the communication request via the service providers addressing the plurality of questions via the communication platform;
electronically determining behavior metrics associated with each of the plurality of service providers including a response time value based on a timeliness of the response by the service provider to the communication request and a response quality value based on a completeness of the response by the service provider to the communication request;
receiving a medical care inquiry regarding the patient the medical care inquiry requesting medical services for the patient;
accessing a third party database and retrieving third party information therefrom;
electronically generating a quality assurance value for each of the plurality of service providers, relative to the medical care inquiry, based at least on the behavior metrics;
electronically generating recommendations relating to the service providers based on the quality assurance value and the third party information and
transmitting the recommendations including-a ranking of the quality assurance values associated with each of the service providers, the rankings visible within a user interface executed on a third party processing device.

15. The method of claim 14, wherein the communication request includes an electronic question submitted to a first service provider of the plurality of service providers across the communication platform from a second service provider of the plurality of service providers.

16. (canceled)

17. The method of claim 14, wherein the plurality of questions provide for a root cause analysis of the medical care incident.

18. (canceled)

19. The method of claim 14 further comprising:
collecting patient data from a plurality of data sources and storing the patient data within a healthcare database, wherein one of the data sources is an electronic medical record of the patient; and
generating the quality assurance values also based on the patient data.

20. (canceled)

21. The method of claim 1, the medical care inquiry is received from a second service provider, the method further comprising:
receiving a referral request from the second service provider, the referral request including the medical care inquiry regarding the patient; and
electronically transmitting a referral response to the second service provider including the recommendation noted therein.

22. The system of claim 9, the medical care inquiry is received from a second service provider, the processing device further operative to:
receive a referral request from the second service provider, the referral request including the medical care inquiry regarding the patient; and
electronically transmit a referral response to the second service provider including the recommendation noted therein.

23. The method of claim 14, the medical care inquiry is received from requesting service provider, which is one of the plurality of service providers, the method further comprising:
receiving a referral request from the requesting service provider, the referral request including the medical care inquiry regarding the patient; and
electronically transmitting a referral response to the requesting service provider including the recommendation noted therein.
